(19) United States
(12) Patent Application Publication
(10) Pub. No.: US 2012/0172487 A1
(43) Pub. Date: Jul. 5, 2012

(54) Artificial bone tissue and related methods

(76) Inventor: Jeffrey A. McKinney, Lafayette, CA (US)

(21) Appl. No.: 13/374,556

(22) Filed: Jan. 3, 2012

Related U.S. Application Data
(60) Provisional application No. 61/460,468, filed on Jan. 3, 2011.

(51) Int. Cl. A61F 2/28

(52) U.S. Cl. 523/115

(57) ABSTRACT

The present invention generally relates to an artificial bone composition, methods of making the composition, and methods of using the artificial bone. In one aspect, the present invention is directed to a composition for inclusion in artificial bone. The composition includes hydroxyapatite and between 0.5 weight percent and 1.5 weight percent of a polymer. The composition has a microstructure substantially similar to a marine carbonate skeleton.

This application claims priority under 35 U.S.C. §119(e) to U.S. provisional patent application Ser. No. 61/460,468, filed Jan. 3, 2011, which is hereby incorporated by reference.

### FIELD OF THE INVENTION

The present invention generally relates to an artificial bone composition, methods of making the composition, and methods of using the artificial bone.

### BACKGROUND OF THE INVENTION

Bone is the second most transplanted material in patients, with only blood transfusions being performed more frequently. Researchers have conducted extensive work directed toward the development of an artificial bone composition and structure that lessens host rejection and provides for replacement by natural bone. Examples of recent work in the area are summarized below.

U.S. Pat. Appl. 2010/0312375 discusses an artificial bone forming method. The method involves the following steps: a) a powder layer forming step for forming, into a flat powder layer, a powder bone material having biocompatibility and hardening by hydration, b) a partial hardening step for jetting an aqueous solution with biocompatibility to a part of the powder layer to harden a jetted portion by hydration, and c) an artificial bone forming step for repeating the steps a) and b) for lamination to form a specified artificial bone of a predetermined three-dimensional structure in which the hardened portions are connected to each other.

U.S. Pat. Appl. 2010/0222883 reports an artificial bone with substantially unidirectionally-oriented pores in the inside of an artificial bone material (artificial bone main body). On its surface it has a marker showing the orientation direction of the substantially unidirectionally-oriented pores. The marker may be a line symbol, a protrusion, a pit, etc. The direction of substantially unidirectionally-oriented pores in an artificial bone can be easily confirmed.

U.S. Pat. Appl. 2010/0166828 discusses a method for producing artificial bone. The method involves the following steps: a) overlapping an apatite/collagen composite gel and a collagen gel and freeze-drying them to form a two-layer, porous body integrally comprising an apatite/collagen composite layer and a collagen layer; and, b) compressing the two-layer, porous body with a monoaxial press before cross-linking. The apatite/collagen composite is preferably apatite-rich for bone formation, and a weight ratio of apatite to collagen in the apatite/collagen composite is preferably 6/4 to 9/1. The sheet preferably includes pores that are 100-1000 μm in diameter at a density of 1 or more per 1 cm².

U.S. Pat. Appl. 2009/0317278 reports an artificial bone that is easy to bond to a living bone and has a mechanical property approximate to that of a living bone. The artificial bone includes: a dense part made of titanium or a titanium alloy, in the shape of a frame that is approximate to a part of an outer face of a living bone; and a porous part made of sintered particles of titanium or a titanium alloy having the same or different composition as the titanium alloy used for the dense part. The porous part has a porosity of 40% or more, and the dense part and the particles of the porous part are sintered to each other at an interface.

The extensive research performed in the artificial bone area has not provided a material that generally meets patient requirements. There is accordingly a need for new compositions and methods.

### SUMMARY OF THE INVENTION

The present invention generally relates to an artificial bone composition, methods of making the composition, and methods of using the artificial bone.

In one aspect, the present invention is directed to a composition for inclusion in artificial bone. The composition includes hydroxyapatite and between 0.5 weight percent and 1.5 weight percent of a polymer. The composition has a microstructure substantially similar to a marine carbonate skeleton.

### DETAILED DESCRIPTION OF THE INVENTION

The present invention is directed to compositions and methods relating to artificial bone. The composition includes one or more polymers, which improve the physical properties of the material as compared to the same composition that does not include the polymer(s). Polymers included in the composition are naturally occurring (e.g., obtained through purification from a living organism), synthetic (i.e., obtained through polymerization of one or more monomers in a chemical laboratory), semi-synthetic (i.e., obtained through one or more chemical reactions performed on a naturally occurring polymer), or semi-natural (i.e., obtained by introducing synthetic materials into an organism and subsequently purifying a polymer from the organism that contains the synthetic materials).

The polymers bind calcium and/or phosphate. Where the polymer is a naturally occurring calcium binder, it typically has an apparent association constant (K_{A}) greater than 500 M⁻¹, as measure by the method of Hunter (Example 1). In some cases, the polymer has a K_{A} greater than 700 M⁻¹, greater than 1,000 M⁻¹, greater than 2,000 M⁻¹, greater than 3,000 M⁻¹, or greater than 5,000 M⁻¹. The polymer, in other cases, has a K_{A} greater than 10,000 M⁻¹, 20,000 M⁻¹, or 30,000 M⁻¹.

Where the polymer is a synthetic calcium binder, it typically has a binding constant (pKe) relative to calcium greater than 3.0, as measured by the method of Itaconix (Example 2). In some cases, the polymer has a pKe greater than 3.5, 4.0, 4.5, or 5.0. The polymer, in other cases, has a pKe greater than 5.5, 6.0, 6.5, or 7.0.

The polymer typically has a molecular weight less than 50,000 g/m. In some cases, the polymer has a molecular weight less than 45,000 g/m, less than 40,000 g/m, less than 35,000 g/m, less than 30,000 g/m, less than 25,000 g/m, less than 20,000 g/m, less than 15,000 g/m or less than 10,000 g/m. The polymer, in other cases, has a molecular weight less than 9,000 g/m, 8,000 g/m, 7,000 g/m, 6,000 g/m, or 5,000 g/m.

Nonlimiting examples of polymers used in the composition of the present invention include: polyphosphate; chondroitin, including chondroitin sulfate; hyaluronic acid; heparin; keratin sulfate; polyitaconic acid; and, polyacrylic acid.

In addition to the polymer, compositions according to the present invention typically include calcium and phosphate in the form of hydroxyapatite.

The composition is typically included in or on a material having a microporous structure. The microporous structure may be either naturally occurring (e.g., skeletal material of marine life) or synthetic (e.g., ceramic, metal or metal alloy). In one example, the microporous structure is a microporous carbonate skeletal source from marine life (e.g., coral, such as Porites, or echinoderm spine material, such as Acathaster planci).

Where the structure is from a carbonate skeletal source, the artificial bone is made through an exchange reaction. The skeletal carbonate is exchanged with phosphate, which effectively creates a synthetic phosphate (e.g., hydroxyapatite or whitlockite) having substantially the same microstructure as the skeletal source. See U.S. Pat. No. 3,929,971, which is incorporated-by-reference for all purposes.

The exchange reaction is typically a hydrothermal chemical exchange reaction. Reaction temperature of the exchange is typically between 100° C. and 600° C., and reaction pressure is typically between 1,500 psi and 75,000 psi. The reaction is oftentimes complete within 1 hour to 14 days, depending upon reaction temperature, pressure and reactant structure.

At least the following reactant types are included in the exchange reaction: a substantially water soluble phosphate and a polymer according to the present invention. The reaction is usually carried out in water. The amount of phosphate included in the water is of any suitable value, but one ratio of carbonate skeletal material to phosphate to water (by weight) is 1:1:4. The amount of polymer in the reaction mixture is such that less than 5.0 percent of the polymer by weight is included in the artificial bone composition. In some cases, between 4.0 percent and 0.1 percent, between 3.5 percent and 0.2 percent, between 3.0 percent and 0.3 percent, between 2.0 percent and 0.4 percent, or between 1.5 percent and 0.5 percent of the polymer is included in the composition.

Any suitable phosphate that is substantially water soluble may be employed as the phosphate contributing reactant in the hydrothermal chemical exchange reaction. Examples of phosphates include, without limitation, alkali metal phosphates, ammonium orthophosphates (including the acid phosphates and mixed phosphates), calcium orthophosphates, acid phosphates, as well as orthophosphoric acid (including its hydrates and derivatives).

Orthophosphates and acid phosphates used to make the composition of the present invention include, without limitation, Li₃(PO₄), LiH₂(PO₄), Na₃(PO₄), Na₂H(PO₄), Na₃H₃(PO₄)₂, NaH₂(PO₄), Na₄H₅(PO₄)₃, NaH₅(PO₄)₂, K₃(PO₄), K₂HPO₄, K₇H₅(PO₄)₄, K₅H₄(PO₄)₃, KH₂(PO₄), KH₅(PO₄)₂, (NH₄)₃PO₄, (NH₄)₂HPO₄, NH₄H₂(PO₄), NH₄H₅(PO₄)₂, NH₄H₈(PO₄)₂, 2CaO.P₂O₅, CaHPO₄, Ca₄P₂O₉, Ca(H₂PO₄)₂ and CaO.P₂O₅ their hydrates and mixed salts.

### EXAMPLES

### Example 1

### Hunter Method for Determining Apparent Association Constants

Materials. Pronase (protease type XIV) and chondroitinase ABC were obtained from Sigma Chemical Co. ⁴⁵CaCl₂ and Na₂³⁵SO₄ (SJS-IA, 25-40 Ci/mg) were obtained from Amersham International. Chondroitin I-sulfate (type A, from whale cartilage), heparin (Grade I, from porcine intestinal mucosa), and hyaluronic acid (Grade IV, from bovine vitreous humour) were obtained from Sigma Chemical Co.

Purification of glycosaminoglycans. Keratan sulfate was purified from human costal cartilage by the method of Mathews. Cartilage from cadavers aged 40 years or older was dissected free of periosteum and soft tissue, then sliced finely. Following lyophilization, cartilage was incubated at approximately 5 mg/ml in 0.2 M Tris-HCl, pH 7.4, containing 2 mg/ml Pronase at 60° C. for 48 h. An insoluble residue was removed by centrifugation, and the supernatant was added to 1.25 vol of absolute ethanol.

After standing at 4° C. overnight, the precipitate (containing mostly CS) was removed by centrifugation, and the ethanol supernatant was concentrated by evaporation at 60° C. Following dialysis against distilled water, an aliquot of cartilage extract equivalent to 1 g of GAG was applied to a 26×100-cm column of Dowex 1×2 (200-400 mesh), and eluted sequentially with water, 1.5, 2.0, 3.0, and 5.0 M NaCl. Eluates were concentrated by ultrafiltration using an Amicon PM-10 membrane, dialyzed versus distilled water, and lyophilized. The 3.0 and 5.0 M fractions contained essentially pure KS (15). The 3.0 M fraction was used in the experiments described below.

Commercial GAG preparations were dialyzed for 24 h against distilled water, then lyophilized.

Equilibrium dialysis. Dialysis tubing (10 mm diameter, 10,000 Af, nominal cutoff) was soaked in 0.1 M HCl at 4° C. for 48 h prior to use, then washed extensively with deionized water. To determine the effect of GAG concentration on Ca binding, 1-ml aliquots of CS, KS, and HA (0.5-2.0 mg/ml in dH₂O) were dialyzed against 1 liter of 1 mM CaCl₂/0.02 μCi/ml ⁴⁵CaCl₂/20 mM Tris-HCl, pH 7.4. To determine the effect of NaCl on Ca binding to CS, 1-ml aliquots of CS (2 mg/ml in dHzO) were dialyzed against 200 ml of the same solution containing 0-140 mM NaCl. In both cases, dialysis was for 24 h at room temperature, and 0.5-ml aliquots of dialysates and bulk solution were removed for analysis of ⁴⁵Ca activity by liquid scintillation counting. For quantitative analysis of Ca binding to GAGS, 1-ml aliquots of CS, KS, HA, and heparin (1 mg/ml in 10 mM Tris-HCl, pH. 7.4) were dialyzed against 29 ml of Tris buffer containing either CaCl₂ or Na₂SO₄ in the range 0.05-10 mM plus either ⁴⁵CaCl₂ or Na₂³⁵SO₄ in the range 0.001-0.2 μCi/ml. Following rotary mixing at room temperature for 24 h, 0.5 ml aliquots of GAG solution and bulk solution were removed for liquid scintillation counting. ⁴⁵Ca and ³⁵S dpm values were converted to concentration values by counting aliquots of CaCl₂/⁴⁵Ca and Na₂SO₄/³⁵S stock solutions. “Bound” Ca was calculated by subtraction of concentrations inside and outside the dialysis membrane. “Excluded” SO₄ was calculated similarly.

Analysis of binding data. In order to perform quantitative analysis of binding of Ca to GAGS, it is necessary to correct apparent binding data for Gibbs-Donnan effects. This was achieved by measuring the exclusion of the divalent anion, sulfate. In the system used in this study, a small ionic species is distributed between two compartments, one of which contains a macromolecule (M) bearing z negative charges. For Ca²⁺, the equilibrium concentrations in the two compartments (i and o) are related to the charges on the macromolecule as follows:

[Ca]ᵢ/[Ca]₀=−z·[M]/4[Ca]₀+square root of [(z·[M]/4[Ca]₀)2+1]

The corresponding relationship for SO₄²⁻ is

[SO₄]₀/[SO₄]ᵢ=−z·[M]/4[SO₄]ᵢ+square root of [(z·[M]/₄[SO₄]ᵢ)²+1]

At equivalent concentrations of [Ca] and [SO₄], and if z and [M] are constant

[Ca]ᵢ/[Ca]₀=[SO₄]₀[SO₄]ᵢ

Therefore, the distribution of SO₄ may be used to correct the apparent binding of Ca to GAGs for Gibbs-Donnan effects. As the Ca-GAG equilibrium is established inside the dialysis membrane, it is also necessary to correct the free Ca values measured outside the membrane to account for Gibbs-Donnan effects. This was achieved using the ratio of SO₄ across the membrane (typically approximately 1.06).

Binding data were analyzed by the method of Scatchard. The equation describing the binding of a monovalent ligand to a macromolecule with one class of noninteracting sites is

[B]/[F]=−K_{A}·[B]+n[M]·K_{A}

where [B] and [F] are the bound and free ligand concentrations, respectively. K_{A} is the apparent association constant, [M] is the concentration of macromolecule, and n is the number of binding sites per macromolecule. The negative of the slope is therefore K_{A}, and n is calculated from the x-intercept. Hunter et al., Archives of Biochemistry and Biophysics Vol. 260, No. 1, pp. 161-167, 1988.

### Example 2

### Itaconix Method for Measuring Binding Constants

A calcium selective electrode was calibrated with standards. A known amount of polymer was titrated with calcium chloride (10%) in a stirred vial. Tests were performed at pH 9.7, 50° C.

An ion selective electrode is used to determine the quantity of free calcium. [Ca_{bound}] is the concentration of calcium bound (known from the quantity of calcium free and the quantity of calcium bound by the polymer), [Ca_{free}] is the concentration of free calcium directly given by the electrode and [Polymer_{free}] is the concentration of polymer not bound with calcium (known with the quantity of polymer introduced at the beginning and the quantity of calcium bound). The quantity pKe can be determined as a function of repeat unit.

pKe=log([Ca_{bound]/[}Ca_{free}][Polymer_{free}]

See, www.itaconix.com/ . . . /Application%20002%20%20Calcium%20Binding%20V2.pdf.

### Example 3

### Roy Method for PreparingPorous Biomaterials

Slices of massive scleractinian coral Porites and spines of the asteroid Acanthaster planci were used as starting materials to provide aragonite and calcite polymorphs of calcium carbonate, respectively. Hydrothermal techniques were employed for the chemical exchange of these carbonate materials with a phosphate. Sections of Porites coral and of Acanthaster planci spine together with weighed quantities of reactants and water (the source carbonate material being completely immersed in the resulting aqueous solution) were sealed in a gold tube, heated at elevated temperatures and pressures for periods of time varying from 12 hours to one week and the resulting reaction product cooled and examined. Upon examination it was found that essentially complete replacement of aragonite by phosphate materials had been achieved. The porous interconnecting structures of the source materials were preserved. For example, hydroxyapatite replaced original Porites aragonite carbonate and preserved its structure. Typical experimental conditions for exchange reactions carried out to produce hydroxyapatite and whitlockite are: coral, reactant (NH₄)₂HPO₄, temperature between 180° C. and 350° C., 15,000 psi, 12 to 48 hours; coral, reactant (NH₄)₂HPO₄+Ca(OH)₂, temperature between 250° C. and 350° C., 15,000 psi, 24 to 48 hours; spine, reactant (NH₄)₂HPO₄, temperature between 260° C., 8,000 to 15,000 psi, 24 hours; spine, reactant (NH₄)₂HPO₄+Ca(OH)₂, temperature between 260° C., 15,000 psi, 24 hours. See, U.S. Pat. No. 3,929,971, which is incorporated-by-reference for all purposes.

1. A composition for inclusion in artificial bone, wherein the composition comprises hydroxyapatite and a polymer, and wherein the polymer comprises between 0.5 weight percent and 1.5 weight percent of the composition, and wherein the composition has a microstructure substantially similar to a marine carbonate skeleton.

2. The composition according to claim 1, wherein the polymer binds calcium, and wherein the apparent association constant is greater than 500 M⁻¹.

3. The composition according to claim 1, wherein the polymer binds phosphate.

4. The composition according to claim 1, wherein the polymer binds calcium and phosphate, and wherein the apparent association constant for calcium is greater than 500 M⁻¹.

5. The composition according to claim 1, wherein the polymer is a naturally occurring calcium binder, and wherein the apparent association constant for calcium is greater than 500 M⁻¹.

6. The composition according to claim 1, wherein the polymer is a synthetic calcium binder, and wherein it has a binding constant relative to calcium greater than 3.0.

7. The composition according to claim 1, wherein the polymer has a molecular weight less than 50,000 g/m.

8. The composition according to claim 1, wherein the polymer is selected from a group consisting of polyphosphate, chondroitin, hyaluronic acid, heparin, keratin sulfate, polyitaconic acid and polyacrylic acid.

9. The composition according to claim 8, wherein the polymer has a molecular weight less than 50,000 g/m.

10. The composition according to claim 9, wherein the polymer has a molecular weight less than 20,000 g/m.
